(19) United States
(12) Patent Application Publication
(10) Pub. No.: US 2009/0124551 A1
(43) Pub. Date: May 14, 2009

(54) METHODS OF ADMINISTERING BONE HEALTH COMPOSITIONS DERIVED FROM MILK

(75) Inventors: Ian Reginald Reid, Auckland (NZ); Jillian Cornish, Auckland (NZ); Neill Ward Haggarty, Palmerston North (NZ); Kay Patricia Palmano, Palmerston North (NZ)

Correspondence Address: KNOBBE MARTENS OLSON & BEAR LLP, 2040 MAIN STREET, FOURTEENTH FLOOR, IRVINE, CA, 92614, US

(73) Assignee: NEW ZEALAND DAIRY BOARD, Wellington (NZ)

(21) Appl. No.: 12/351,616

(22) Filed: Jan. 9, 2009

Related U.S. Application Data
(62) Division of application No. 10/398,628, filed on Oct. 10, 2003.

(30) Foreign Application Priority Data
Oct. 5, 2000 (NZ) 507335
Sep. 27, 2001 (NZ) PCT/NZ01/00200

(51) Int. Cl. A61K 38/16; A61K 31/7088; A61P 19/00

(52) U.S. Cl. 514/12; 514/44

(57) ABSTRACT

The invention relates to bone health compositions comprising an acidic protein fraction of milk, to a method of producing said bone health composition, to methods of treatment comprising said bone health compositions and to medicinal uses of said bone health compositions. One broad aspect of the invention provides a bone health composition comprising an acidic protein fraction derived from milk, from a component of milk, from whey, from hydrolysates thereof, or from a combination thereof wherein the composition does not comprise caseinoglycomacropeptide (CGMP). Another broad aspect provides a method of manufacturing the composition of the invention using anion exchange chromatography.

### REFERENCE TO RELATED APPLICATIONS

The present application claims priority under 35 U.S.C. § 120 as a divisional of U.S. patent application Ser. No. 10/398,628, filed Oct. 10, 2003 and currently pending, which is incorporated by reference in its entirety and is in turn the U.S. National Phase of PCT/NZ01/00200, filed Sep. 27, 2001 and claims priority under 35 U.S.C. § 119 to New Zealand Patent Application Number 507335, filed Oct. 5, 2000.

### FIELD OF THE INVENTION

The invention relates to bone health compositions derived from an acidic protein fraction of milk and in particular to bone health compositions derived from an acidic protein fraction of whey, to a method of producing said bone health compositions, to methods of treatment comprising said bone health compositions and to medicinal uses of said bone health compositions.

### BACKGROUND TO THE INVENTION

### Bone Physiology and Disease

One of the most prevalent and costly bone diseases is osteoporosis, characterised by a gradual thinning and weakening of the bones. If this deterioration goes untreated, bones are likely to break or fracture with very little trauma.

Although the process of bone loss begins gradually in the mid to late thirties, it is so slow that it may take many years before a sufferer becomes aware of it. Women, generally, are at greater risk of developing osteoporosis than men. This is because, following the menopause, women experience a rapid loss of bone from the skeleton due to the decrease in estrogen production.

Until a person is around 40, the process of breaking down (resorption) and building up (reforming) bone by osteoclasts and osteoblasts respectively is a nearly perfectly coupled system, with one phase stimulating the other. Bone comprises an extracellular protein matrix (mostly collagen fibrils) interspersed with bone cells (osteocytes) with a mineral component laid on to this consisting of calcium salts and other minerals including sodium, magnesium and fluoride. Osteoclasts resorb bone at a particular site and then undergo programmed cell death. Osteoblasts replace the protein matrix (osteoid) and mediate its remineralisation. During remineralisation, the osteoblasts are encased within the calcified material and become osteocytes, cells that help maintain the structure of the bone. Bone turnover mediated by the osteoclasts and osteoblasts occurs throughout life and is known as “remodelling”. Estrogen deficiency is believed to delay the programmed cell death or apoptosis of osteoclasts thus leading to a net bone loss.

As a person ages, the remodelling system breaks down and the two processes (resorption and reformation) become out of synchronisation. The reasons for this are not clear. Some individuals have a very high turnover rate of bone; some have a very gradual turnover, but the breakdown of bone eventually overtakes the build-up. Because the patterns of reforming and resorbing bone often vary from patient to patient, experts believe a number of different factors account for this problem. Important hormones, such as estrogen, parathyroid hormone, vitamin D, and blood factors that affect cell growth are involved in this process. Changes in the levels of any of these factors could play a role in the development of osteoporosis.

Post-menopausal women often undergo Hormone Replacement Therapy (HRT) to compensate for reducing natural estrogen levels. HRT is usually not prescribed before a patient is very close to the critical limit of bone loss that can lead to osteoporotic fractures.

Since calcium is an essential ingredient of bone, it is believed necessary to have adequate calcium intake either in the diet or in supplements. Without Vitamin D the calcium cannot be incorporated into the bone and therefore adequate Vitamin D intake is needed too.

### Milk and Milk Proteins

As well as being a good protein source, bovine milk is often the main dietary source of calcium and vitamin D. Milk proteins and their effects on bone disease have been the focus of a large amount of research. The proteins found in milk include immunoglobulins, growth factors, bovine serum albumin (BSA), alpha-lactalbumin, beta-lactoglobulin and a large number of caseins, all of which are phosphoproteins. These proteins, with the exception of casein, are also present in whey. Milk is known to contain a variety of mitogenic proteins and proteins which may be involved directly in bone remodelling.

Caseinoglycomacropeptide (CGMP) is a peptide released from kappa-casein during the rennet-mediated casein coagulation step (through the action of chymosin) of the cheese making process and is found in the whey fraction which is known as Sweet Whey or Cheese Whey. CGMP is sometimes referred to simply as GMP (glycomacropeptide). Cheese whey proteins consist of 15% to 20% CGMP. CGMP has been put forward as one of the bone health promoting components of milk, as disclosed in WO 00/49885 (discussed below).

Lactic acid whey is produced by fermentation with lactic acid bacteria or direct addition of lactic acid during the manufacture of caseinate or cottage and ricotta cheeses. Mineral acid whey is produced by addition of mineral acids during caseinate manufacture. Lactic acid whey and mineral acid whey do not contain CGMP. The basis of these two processes is to lower pH to about 4.6 to cause casein to precipitate as opposed to using the action of chymosin to cause precipitation.

Therefore any milk products that have not been exposed to chymosin will not contain CGMP.

Growth factors (IGF—Insulin-like Growth Factor, TGF—Transforming Growth Factor etc), immunoglobulins, BSA and some beta-lactoglobulin are recovered from milk or whey by cation exchange chromatography. Some growth factors are recovered as neutral proteins. CGMP is an acidic protein fraction recoverable by anion exchange.

Osteopontin (OPN) is a highly phosphorylated and glycosylated protein found in all body fluids (including milk) and in the extracellular matrix of mineralized tissues. OPN, one of the more abundant non-collagenous proteins in bone, is localized to cell-matrix and matrix-matrix interfaces in mineralized tissues, where it is deposited as the result of osteoclast action. OPN may protect the exposed bone surface or prime it for subsequent cell-matrix interactions. It has been proposed that OPN acts as an opsonin, facilitating macrophage adhesion and phagocytosis of particulate mineralized tissue debris. OPN can be cross linked by transglutaminase, and it can bind to various extracellular molecules including type I collagen, fibronectin and osteocalcin. This might be expected to add physical strength to extracellular matrices. OPN appears to promote the attachment of bone cells to bone matrix. OPN is present in vertebrate blood serum at relatively high concentrations and is thus also excreted in mammalian milk. (Denhardt, D. T. and Noda, M., Osteopontin expression and function: Role in bone remodelling, J. Cell Biochem. Suppl. 30/31:92-102 (1998)).

### PRIOR ART

The majority of published patents in the prior art have focused on the bone growth activity of basic proteins—i.e., those derived by the use of cation exchange.

New Zealand Patent Specification 503608 teaches of preparing a bone anti-resorption agent from milk or heated whey by cation exchange chromatography. Cystatin or enzyme hydrolysed products of cystatin are disclosed as being effective in suppressing bone resorption. Bone, bone and joint and periodontal disease may be prevented (or treated) by ingesting drinks, food products or feeds in which cystatin or its hydrolysed products are present.

New Zealand Patent Specification 282898 discloses bovine IGF-1 like growth factors that are purified by cation exchange chromatography from milk, skim milk, cheese whey, reconstituted whey, WPC, WPI, milk powder, whey powders or colostrum. These materials are preferably heated before the cation exchange step. The binding is done at a temperature between 4 and 40 degrees C. using a defined protein to cation exchanger ratio. The growth promoting effects of the preparation were demonstrated in cultured osteoblast like cells (MC3T3-E1 cells). The composition is claimed to be useful for preventing or treating bone and articulation diseases, in particular osteoporosis. If administered to humans during their growth phase, their peak bone mass may be increased. As a raw ingredient, the claimed material is useful for incorporation in beverages, foods medicines and animal diets.

European Patent Specification EP 0787499 (corresponding to Japanese Patent Abstract 8045566) teaches that kininogen, found in bovine plasma and milk, promotes bone formation and inhibits bone resorption. Whole kininogen, the fragment 1.2 of kininogen and the enzymatically degraded products of kininogen (molecular weight range 0.1-70 kDa (kilo Daltons) are all claimed to be active. The patent covers uses in drinks, foods, medicines and feed for this family of products. A weakly basic cation exchanger is used to prepare kininogen.

New Zealand Patent Specification 314286 discloses N-terminal sequences of the High Mobility Group (HMG) protein and amphoterin, or their degradation products as bone-growth promoters and bone resorption inhibitors. The degradation products that are active have a molecular weight of 0.1-20 kDa. The claimed preparations can be used as components of food, drink, medicine or feed where calcium is included. HMG protein and amphotericin recovered from other body fluids also acts similarly. In the example given the HMG protein of milk is isolated by cation exchange chromatography and subsequently purified on an S-Sepharose and a Mono-Q column.

New Zealand Patent Specification 246211 teaches of an osteoblast growth/bone enhancing factor from whey that is obtained by precipitating acidified whey with ethanol and having a molecular weight of between 5-28 kDa. The active agent is recovered as a water extract of the ethanol-precipitated fraction. Alternately, the same factor can be recovered in the permeate when heated whey is ultrafiltered through a 30 kDa membrane. Isoelectric point of the preparation is between 4 and 9. No anion exchange chromatography is used.

New Zealand Patent Specification 314097 discloses a milk derived protein of molecular weight between 2 and 24 kDa with an isoelectric point of between 7.5-11.0 having an osteoblast proliferation effect, a bone strengthening effect and a bone resorption inhibiting effect. The active protein is recovered by cation exchange chromatography and is eluted with 0.1M-1.0M salt. Also claimed are food, drink, medicines and feeds containing such a preparation.

New Zealand Patent Specification 301362 (corresponding to Japanese Patent Abstract JP 207508) teaches of an osteoclast inhibiting protein, the DNA encoding it and a method for expression of the DNA. The protein is 60 kDa under reducing conditions and from 60-120 kDa under non-reducing conditions. The protein can be purified by cation exchange or by binding to a heparin column. The biological activity of the protein is decreased by heating at 56 degrees C. for 10 minutes.

European Patent Specification EP 704218 discloses the preparation of a basic milk based protein fraction and a milk based basic peptide fraction. These are derived by cation exchange chromatography of milk or whey. Both products, and their hydrolysates, promote bone growth and suppress the resorption of osteoclasts when orally administered. Can be presented as a food or a drink product. The compositions described are claimed to be useful for treating or preventing various bone diseases such as osteoporosis.

PCT Patent Specification WO 00/49885 discloses a composition for prevention or treatment of a bone or dental disorder which comprises a milk protein hydrolysate. In preferred embodiments the milk protein hydrolysate is a hydrolysate of casein, in particular a caseinoglycomacropeptide (CGMP), a mimetic, homologue or fragment thereof in a bioavailable form which retains the ability of CGMP to inhibit bone resorption or bone loss; or favour calcium absorption, retention or calcification; or a combination thereof. The composition is produced from sweet whey by concentration and weak anion chromatography.

Bayless et al (Isolation and biological properties of osteopontin from bovine milk, Protein Expression and Purification 9(3): 309-314 (1997)) disclose a method for purifying osteopontin present in raw skim milk using DEAE-Sephacel (at the natural milk pH of 6.6) mixed overnight at 4 degrees C. The unbound fraction was removed and other bound proteins were removed with a 0.25M NaCl wash. An osteopontin containing fraction was recovered by a 0.3M elution. Osteopontin containing fractions were pooled, made 4M in salt and then purified by hydrophobic interaction chromatography on a phenyl Sepharose column (twice). Isolation from raw skim is not acceptable commercially and the focus is on a highly purified sample, not an enriched product stream.

An early study by Takada et al (Milk whey protein enhances the bone breaking force in ovariectomised rats, Nutrition Research 17, 1709-1720 (1997)) shows that bone strengthening components are present in whey. The active components are heat stable and are present in the low molecular weight, 30-70% ethanol-precipitable portion of whey. No fractionation of whey was done.

Yun S. S. et al (Isolation of mitogenic glycophosphopeptides from cheese whey-protein concentrate, Bioscience Biotechnology and Biochemistry, 60(3): 429-433 (1996)) investigated the immunological function of cheese whey protein concentrate (CWPC) using mitogenic activity in murine splenocytes as an index. A fraction isolated by gel filtration and anion exchange chromatography of CWPC showed high mitogenic activity. The study's results demonstrated that cheese whey contains a glycophosphopeptide (GPP) having strong mitogenic activity.

Sorensen E. S. et al (Purification and characterization of 3 proteins isolated from the proteose peptone fraction of bovine-milk, Journal of Dairy Research, 60(2):189-197 (1993)) isolated three major proteins from the proteose peptone of bovine milk. These were purified by Sephadex G-75 gel chromatography, Q-Sepharose ion-exchange and additional Sephadex G-75 gel chromatography in the presence of urea. From their mobility in a gradient SDS-PAGE the proteins were found to have molecular masses of 17, 28 and 60 kDa. The N-terminal amino acid sequence of the 17 kDa protein was found to be homologous with a camel whey protein. This protein had not previously been described in bovine milk. From the SDS-PAGE results, the 28 kDa protein was judged to be the major protein of proteose peptone, contributing approximately 25% of the total. The N-terminal amino acid sequence showed no homology to any known protein sequence, but the amino acid composition indicated that the 28 kDa protein is identical with the PP3 component from the proteose peptone fraction of bovine milk or part of it. The 60 kDa protein was found to be bovine osteopontin, a very highly phosphorylated protein with an Arg-Gly-Asp sequence which mediates cell attachment.

Publications in the science and the patent literature have not shown that acidic fractions of milk (or whey) provide a (potential) source of a bone anti-resorption agent.

### OBJECTS OF THE INVENTION

It is therefore an object of different aspects of the invention to provide bone health compositions derived from an acidic protein fraction of milk and particularly from an acidic protein fraction of whey; methods of producing such compositions; methods of treatment comprising said compositions; medicinal uses of said compositions; and/or at the least to provide the public with a useful choice.

### SUMMARY OF THE INVENTION

According to one aspect of the invention there is provided a bone health composition suitable for reducing net bone loss comprising an acidic protein fraction of milk, hydrolysates of an acidic protein fraction of milk or a combination thereof wherein the composition does not contain caseinoglycomacropeptide (CGMP).

According to a second aspect of the invention there is provided a bone health composition suitable for reducing net bone loss comprising an acidic protein fraction derived from a component of milk, hydrolysates of an acidic protein fraction derived from a component of milk or a combination thereof wherein the composition does not contain caseinoglycomacropeptide (CGMP).

According to a third aspect of the invention there is provided a bone health composition suitable for reducing net bone loss comprising an acidic protein fraction of whey, hydrolysates of an acidic protein fraction of whey or a combination thereof wherein the composition does not contain caseinoglycomacropeptide (CGMP).

Preferably the compositions of the invention are produced using anion exchange chromatography and more preferably strong anion exchange chromatography.

Preferably a composition of the invention comprises 70% by weight or more of proteins of which 80% by weight or more, and preferably 90% by weight or more, comprise osteopontin and proteose peptones. Preferably the proteose peptones comprise peptides generated from casein by the action of plasmin and include one or more of the proteins selected from the group comprising proteose peptone 5 (PP5), proteose peptone 8-slow (PP8-slow), proteose peptone 8-fast, (PP8-fast), as well as the non-casein proteose peptone 3 (PP3).

Preferably the proteins in a composition of the invention have a molecular weight distribution of 3,000 to 65,000 as measured by SDS-PAGE.

Preferably the sialic acid content of a composition of the invention is in the range 0.8% to 6.5%. Preferably the phosphate content of a composition of the invention is in the range 0.5% to 3%.

Preferably a composition of the invention is derived from any one or more feedstocks selected from the group comprising recombined or fresh whole milk, recombined or fresh skim milk, reconstituted whole or skim milk powder, colostrum, milk protein concentrate (MPC), milk protein isolate (MPI), whey protein isolate (WPI), whey protein concentrate (WPC), whey, reconstituted whey powder, or derived from any milk processing stream, or derived from the permeates obtained by ultrafiltration and/or microfiltration of any one or more of these feedstocks. Preferably the feedstock(s) is obtained from one or a combination of bovine and other dairy sources (for example goat or sheep or other milk-producing mammals). Examples of suitable processing streams include those produced during the manufacture of Lactalbumin™ or TMP™ (Total Milk Protein) isolates. Even more preferably, a composition of the invention is derived from lactic acid whey or mineral acid whey. Methods suitable for the commercial production of whey are described by J G Zadow (Ed) “Whey and Lactose Processing” (Elsevier Applied Science, London and New York, 1992) and T Sienkiewicz and C Riedel (Eds) “Whey and whey utilisation” (Verlag, Germany, 1990).

Preferably a composition of the invention further comprises physiologically acceptable amounts of calcium, magnesium, vitamin C, vitamin D, vitamin E, vitamin K2 and/or zinc.

According to a fourth aspect of the invention there is provided a method of producing a bone health composition comprising the steps of:

(a) providing an aqueous solution that does not contain CGMP and that is derived from any one or more feedstocks selected from the group comprising recombined or fresh whole milk, recombined or fresh skim milk, reconstituted whole or skim milk powder, colostrum, milk protein concentrate (MPC), milk protein isolate (MPI), whey protein isolate (WPI), whey protein concentrate (WPC), whey, reconstituted whey powder, or derived from any milk processing stream, or derived from the permeates obtained by ultrafiltration and/or microfiltration of any one or more of these feedstocks;

(b) subjecting the aqueous solution to anion exchange chromatography at a pH of from about pH 3 to about pH 4.9;

(c) washing the anion exchange medium;

(d) eluting from the anion exchange medium an acidic protein fraction of whey.

Preferably the feedstock(s) is obtained from one or a combination of bovine and other dairy sources (for example goat or sheep or other milk producing mammals).

Preferably the aqueous solution provided in step (a) is lactic acid whey or mineral acid whey. Preferably the aqueous solution provided in step (a) is derived from lactic acid whey or mineral acid whey. Preferably the aqueous solution provided in step (a) comprises hydrolysates of an acidic protein fraction of whey.

Preferably the anion exchange chromatography is strong anion exchange chromatography. Preferably step (b) is carried out between about pH 4 and about pH 4.7, or even more preferably at pH 4.5. Typical examples of the anion exchangers that can be used are the macroporous hydrophilic agarose-based anion exchangers such as Q-Sepharose, Q-Sepharose, Fast Flow, Q-Sepharose Big Beads and Q-Sephadex. Alternately, the cellulose-based macroporous Gibcocel QA Anion exchanger and the Whatman QA Cellulose may be used. Other anion exchangers that can also be used include the polystyrene-based Macropep Q and the Diaion anion exchangers.

Preferably step (c) comprises use of de-mineralised water to wash the medium.

Preferably step (d) is carried out using NaCl or KCl a mixture thereof. Preferably step (d) is carried out using 1 M NaCl. Preferably step (d) is carried out using an acid. Preferably step (d) is carried out using two or more eluting solutions having different pHs. Preferably step (d) is carried out using an eluting solution having a pH between 5.0 and 9.0 and a salt concentration up to 1.0 M.

In a preferred form, a method of the invention further comprises a step or steps before step (a) wherein the step or steps comprise one or more steps selected from the group comprising thermisation, pasteurisation, centrifugation to remove fat, ultrafiltration and/or microfiltration to concentrate the aqueous solution, or reverse osmosis, electrodialysis, or ion exchange chromatography to deionise the preparation.

The composition produced by the method of the fourth aspect of the invention may also comprise calcium, magnesium, vitamin C, vitamin D, vitamin E, vitamin K₂ and/or zinc.

In preferred embodiments of the invention, the bone health compositions of the invention, and those produced by the methods of the invention may be incorporated into dietary supplements, foods or drinks or provided as dietary supplements, food additives or drink additives.

Further preferred embodiments of the invention comprise dietary supplements, nutraceuticals, food additives or drink additives comprising the bone health compositions of the invention, and those produced by the methods of the invention.

Further preferred embodiments of the invention comprise mineral supplements, fortified juice products, cereal or confection bars containing milk, milk powders and milk powder based formulations and products utilising these, UHT and pasturised milks, yoghurts, cultured milks and direct acidified milks, comprising the bone health compositions of the invention, and those produced by the methods of the invention.

In a highly preferred embodiment of the invention, the bone health compositions of the invention, and compositions produced by the methods of the invention comprise one or more compositions selected from the group comprising osteopontin, bone sialoprotein, proteose peptone 3, proteose peptone 5, proteose peptone 8, sialyated and phosphorylated proteins and peptides obtained therefrom, and alpha-s1-casein phosphopeptides.

Preferably the compositions of the invention or compositions produced by the methods of the invention comprise any peptide or peptide fraction within said protein fraction which exhibits beneficial bone health properties.

According to a fifth aspect of the invention there is provided a method of maintaining or improving bone health comprising administering to a patient a composition of the invention or a composition produced by the method of the invention.

According to a sixth aspect of the invention there is provided a method of treating or preventing net bone loss comprising administering to a patient a composition of the invention or a composition produced by the method of the invention.

According to a seventh aspect of the invention there is provided a use of a composition of the invention or a composition produced by the method of the invention in the manufacture of a formulation for maintaining or improving bone health.

According to an eighth aspect of the invention there is provided a use of a composition of the invention or a composition produced by the method of the invention in the manufacture of a formulation for treating or preventing net bone loss.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

The invention consists in the foregoing and also envisages constructions of which the following gives examples.

### BRIEF DESCRIPTION OF THE DRAWINGS

FIG. 1 shows the HPLC Mono Q analysis of the acidic whey protein fraction derived from mineral acid whey in Example 1.

FIG. 2 shows the HPLC Mono Q analysis of the acidic whey protein fraction derived from lactic acid whey in Example 2.

FIG. 3 shows the anti-resorptive effect of the acidic whey protein fraction on mouse calvaria (the vault of the skull) cells in culture from Example 3.

FIG. 4 compares the bone mineral densities of the femurs and spines of the different groups of rats in the in vivo feeding trials from Example 4.

### DETAILED DESCRIPTION OF THE INVENTION

The Applicants have discovered that an acidic protein fraction of milk, of a component of milk, and particularly of whey, can reduce or prevent net bone loss.

The term “acidic protein fraction” is intended to mean a fraction of milk proteins comprising proteins that have an isoelectric point of 4.9 or less.

The acidic protein fraction of the invention has been shown to contain a number of minor acidic whey proteins. These include osteopontin, proteose peptone 3, proteose peptone 5 (PP5), also known as β-casein-5P(f1-105) or β-casein-5P(f1-107), proteose peptone 8-slow (PP8-slow), also known as β-casein-1P(f29-105) or β-casein-1P(f29-107), sialyated and phosphorylated proteins, alpha-s1-casein phosphopeptides, and also a mixture of peptides derived from these proteins by natural proteolysis. Very small amounts of lactosylated alpha- and beta-lactoglobulins, bovine serum albumin and immunoglobulins are also present.

The acidic protein fraction has been shown to contain peptides derived from these proteins generated by the hydrolytic action of the naturally occurring milk protease plasmin. In the specific case of an acidic protein fraction recovered from lactic acid whey, there are a wider range of peptides generated naturally by the action of lactic acid bacterial proteases, as well as by plasmin.

Using an animal model that mimics the effect of menopause on bone (ovariectomised rats—OVX rats), the acidic protein fraction of the invention was found to inhibit the bone resorption normally observed post-menopause that can eventually lead to diseases such as osteoporosis. The acidic protein fraction of the invention is thus useful as a means of treating or preventing diseases such as osteoporosis and osteo-arthritis.

The acidic protein fraction of the invention has an overall sialic acid content in the range of 0.8% to 6.5% and a phosphate content of between 0.5 and 3%.

The acidic protein fraction contains proteins and peptides with a wide range of molecular weights ranging from 3000 Daltons (hydrolysis products) to approximately 65,000 Daltons (proteose peptone 3 aggregates).

Potential feedstocks for commercial recovery of the acidic protein fraction of the invention include recombined or fresh whole milk, recombined or fresh skim milk, reconstituted whole or skim milk powder, colostrum, milk protein concentrate (MPC), milk protein isolate (MPI), whey protein isolate (WPI), whey protein concentrate (WPC), whey, reconstituted whey powder, or derived from any milk processing stream, or derived from the permeates obtained by ultrafiltration and/or microfiltration of any one or more of these feedstocks. Potential feedstocks exclude those derived from sweet whey. Any of these potential feedstocks may be derived from one or more of bovine and other dairy sources (for example goat or sheep or other milk producing mammals). Alternatively, potential feed stocks may be derived from any milk processing stream, such as those produced during the manufacture of Lactalbumin™ or TMP™0 (Total Milk Protein) isolates. Methods suitable for the commercial production of whey are described by J G Zadow (Ed) “Whey and Lactose Processing” (Elsevier Applied Science, London and New York, 1992) and T Sienkiewicz and C Riedel (Eds) “Whey and whey utilisation” (Verlag, Germany, 1990).

An acidic protein fraction of the invention is produced using anion exchange chromatography and preferably strong anion exchange chromatography. Preferably the anion exchange chromatography is carried out at between about pH 3.0 and pH 4.9, more preferably between about pH 4.0 and pH 4.7 and even more preferably at pH 4.5. These pH conditions are optimal for recovery of the acidic protein fraction by anion exchange chromatography because in this range the active component or components are bound to the column and unwanted proteins are almost completely excluded in the unbound fraction. These include major whey proteins such as alpha-lactalbumin, beta-lactoglobulin and bovine serum albumin, which would serve only as diluents of the measured activity.

### INDUSTRIAL APPLICATION

The compositions of the invention and the compositions produced by the methods of the invention may be used for the generation of functional foods by incorporation into food or drink, and for the treatment, management and/or prevention of bone defects (all age groups) including osteo-arthritis, osteoporosis and dental disorders.

It is envisaged that the compositions of the invention will be ingested on a daily basis as neutraceuticals or dietary supplements in order to delay or prevent the onset of debilitating bone disorders.

### EXAMPLES

### Example 1

### Acidic Protein Fraction Derived from Mineral Acid Whey

A 20 L solution of mineral acid whey protein concentrate (Alacen 342—available from NZMP, Wellington, New Zealand) at 10% solids and pH 4.5 was passed through a 2 L column of Q-Sepharose BB (Amrad Pharmacia, Australia) at a flow rate of 110 ml/min. The column was washed with 5 L of demineralised water and eluted with a 1.0M solution of sodium chloride (pH 6.0). Protein adsorption and elution was monitored by measuring the absorbance at 280 nm.

The acidic protein fraction eluted from the column was concentrated approximately 6.25 fold using an Amicon 3K NMCO Spiral ultrafiltration unit (available from Millipore, USA). The concentrated protein retentate was dialysed against water and then freeze dried.

The dry product (56 g recovered) had a content of 79% protein, less than 0.5% calcium, approximately 1.0% phosphorous and 6.0% sialic acid. The amino acid composition of the eluted protein fraction is shown in Table 1.

TABLE 1
Amino acid profile of the acidic protein fraction from mineral
acid whey

| Amino Acid | Content (% w/w) |
|---|---|
| Aspartic acid | 8.19 |
| Serine | 6.22 |
| Glutamic acid | 17.7 |
| Glycine | 1.34 |
| Histidine | 2.22 |
| Arginine | 3.69 |
| Threonine | 4.83 |
| Alanine | 2.59 |
| Proline | 6.15 |
| Tyrosine | 1.88 |
| Valine | 4.18 |
| Lysine | 6.64 |
| Isoleucine | 5.70 |
| Leucine | 6.72 |
| Phenylalanine | 2.86 |

The acidic protein fraction was analysed for whey proteins and proteose peptones by reverse phase HPLC according to the method described by Elgar et al (Journal of Chromatography A, 878 (2000), ppl83-196). An analytical anion exchange Mono Q column HR 5/5 (obtained from Amersham-Pharmacia Biotech, Australia) was used to determine the protein composition of the acidic protein fraction. This detected the presence of osteopontin, alpha-s1-casein phosphopeptides, proteose peptone 3, proteose peptone 5 and beta-lactoglobulin. Proteose peptone 8-slow co-elutes with PP5 so does not appear as a distinct peak. Peptides derived from these proteins by natural proteolysis were also detected, as shown by the broad peaks for each component and the presence of small peaks surrounding the major peaks.

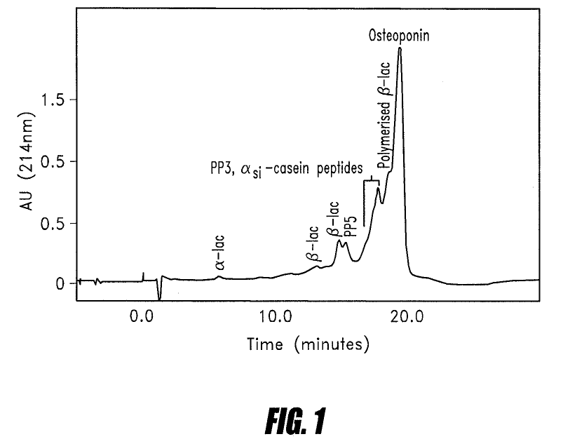

The freeze dried acidic fraction recovered from the anion exchanger was dissolved in 20 mM Tris/HCl buffer, pH 8.0 and loaded onto the Mono Q column. The analytical separation was developed using a triphasic linear gradient to 1M sodium chloride (pH6.0). Protein adsorption and elution was measured at 214 nm. Protein peak identities were resolved using standards prepared in our own laboratory. Standards were prepared for PP5, PP3 and osteopontin. The identity of each standard was confirmed by amino acid sequencing (such as N-terminal sequencing) and their purity by SDS-PAGE and HPLC analysis. Other components were identified by amino acid sequence analysis of peaks trapped from the HPLC run shown in FIG. 1.

The results of these analyses (FIG. 1) showed that osteopontin, alpha-s1-casein fragments, sialyated and/or phosphorylated minor proteins, proteose peptones 5 and 3, and peptides derived from these proteins were present in the acidic protein fraction recovered from mineral acid whey.

### Example 2

### Acidic Protein Fraction Derived from Lactic Acid Whey

A 20 L solution of lactic acid whey protein concentrate (Alacen 312—available from NZMP, Wellington, New Zealand) at 10% solids and pH 4.5 was passed through a 2 L column of Q-Sepharose BB at a flow rate of 110 ml/min. The column was washed with 5 L of demineralised water and eluted with a 1.0M solution of sodium chloride (pH 6.0). Protein adsorption and elution was monitored by measuring the absorbance at 280 nm.

The protein eluted from the column was concentrated approximately 6.25 fold using an Amicon 3K NMCO Spiral ultrafiltration unit (available from Millipore, USA). The concentrated protein retentate was dialysed against water and then freeze dried.

The acidic protein fraction was analysed for whey proteins and proteose peptones by reverse phase HPLC according to the method described by Elgar et al (Journal of Chromatography A, 878 (2000), pp 183-196). An analytical anion exchange Mono Q column HR 5/5 (obtained from Amersham-Pharmacia Biotech, Australia) was used to determine the presence of osteopontin, alpha-s1-casein phosphopeptides, proteose peptone 3 and proteose peptone 5. The presence of peptides derived from these proteins was also observed as discussed in Example 1.

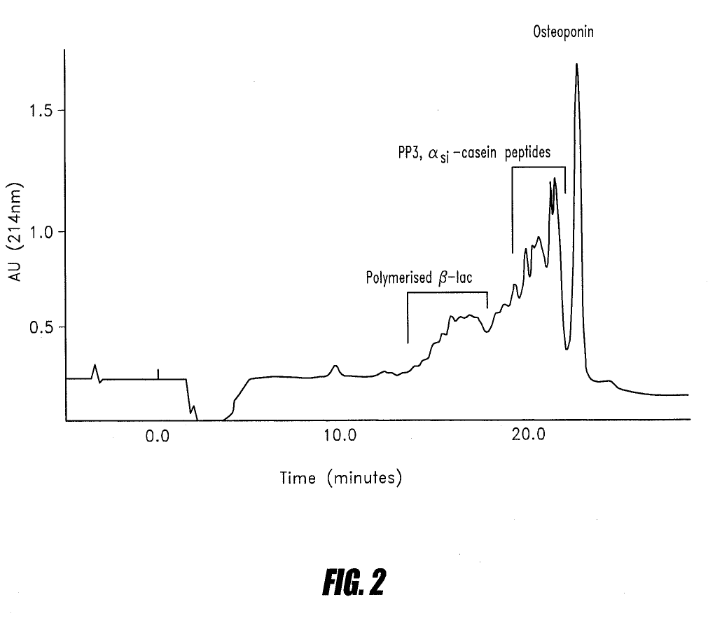

The freeze dried acidic protein fraction recovered from the anion exchanger was dissolved in 20 mM Tris/HCl buffer, pH 8.0 and loaded onto the Mono Q column. The analytical separation was developed using a triphasic linear gradient to 1M sodium chloride (pH 6.0). Protein adsorption and elution was measured at 214 nm. Protein peak identities were resolved using known standards or N-terminal sequencing of trapped peaks. The results of these analyses (FIG. 2) showed that osteopontin, alpha-s1-casein fragments, sialyated and/or phosphorylated minor proteins, proteose peptones 5 and 3 and peptides derived from these proteins, were present in the acid fraction recovered from lactic acid whey. FIG. 2, in comparison with FIG. 1, shows the presence of a larger number of peptides derived from the proteins of the acidic protein fraction.

### Example 3

### In Vitro Analysis of Efficacy

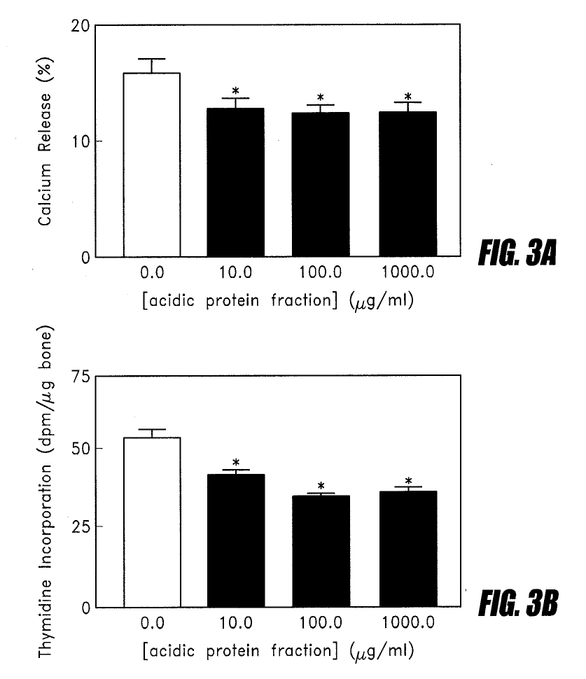

The product from Example 1 was tested in the bone organ culture model as described by Lowe et al (Journal of Bone and Mineral Research (US), 6(12):1277-1283 (1991)). FIG. 3 shows that the acidic protein fraction had anti-resorptive effects on the cells from the bone organ culture at doses as low as 10 ug/ml. Both lower calcium release and lower thymidine incorporation compared to the controls demonstrate the anti-resorptive effect.

### Example 4

### In Vivo Analysis of Efficacy

The ability of the acidic protein fraction from Example 1 to reduce bone loss induced by oestrogen deficiency in the ovariectomised (OVX) rat was studied over a 16 week period. The ovariectomised rat model is a widely accepted model for studying the bone loss that occurs post-menopause.

Thirty 6-month-old female Sprague-Dawley rats were received as 10 Sham-operated animals and 20 OVX animals at age 5.5 months. Sham operated animals undergo anaesthesia and an incision is made but the ovaries are left intact. In the OVX animals the ovaries are removed. On arrival animals were separated into three groups (n=10 per group). These groups are shown in Table 2.

TABLE 2
Treatments used on the three rat groups

| | | Diet plus 0.3% w/w |
|---|---|---|
| | Control Diet | acidic whey protein fraction |
| GROUP A | YES | NO |
| (Sham operated) | | |
| GROUP B | YES | NO |
| (OVX control) | | |
| GROUP C | NO | YES |
| (OVX test) | | |

The animals were separately housed in shoebox cages, and kept in a temperature- (22° C.±2° C.) and light-controlled (12 hour day/night cycle) room. Animals had ad libitum access to deionised water. The animals were fed a balanced semi-synthetic diet consisting of 15% caseinate, 5% cellulose, 5% corn oil, 0.5% calcium, 62% starch and added vitamins and minerals as needed. The casein was adjusted when adding 0.3% (w/w) of the acidic whey protein fraction (sample generated by Example 1). The Sham control group (Group A) and the OVX control group (Group B) received the base diet with no acidic protein fraction added. Group C (OVX rats) received a diet containing 0.3% (w/w) of the acidic protein fraction from Example 1. The daily intake of the animals was measured, and the intake was adjusted weekly according to the SHAM group's body weight in order to prevent body weight gain in the OVX groups. The trial ran for 4 months with monthly measurements.

For bone mineral density (BMD) measurements the rats were scanned every 4 weeks under anasthesia. Rats were weighed and anaesthetised with an appropriate dose level ie. 0.05ml/100 g body weight. The anaesthetic was a mixture of 0.2 ml Acepromazine (ACP)+0.5ml Ketamine+0.1 Xylazine+0.2 ml sterile H₂O, and was administered via an intra-peritoneal injection using a 25 G×⅝″ needle and 1 ml syringe. The rats attained a suitable level of anaesthesia approximately five to ten minutes after injection and remained under anaesthetic for 2 hours.

Bone mineral measurements were taken using a Hologic QDR4000 bone densitometer using a pencil beam unit (Bedford, USA). A daily Quality Control (QC) scan was taken to ensure precision. This was required to meet a coefficient of variation. Regional high-resolution scans were performed using a 0.06 inch (0.1524 cm) diameter collimator with 0.0127 inch (3.23×10⁻² cm) point resolution and 0.0254 inch (6.45×10⁻² cm) line spacing. Rats were placed on an acrylic platform of uniform 1.5 inch (3.81 cm) thickness. Each rat underwent three regional high-resolution scans of the spine and left and right femurs. Rats were positioned supine with right angles between the spine and femur, and femur and tibia.

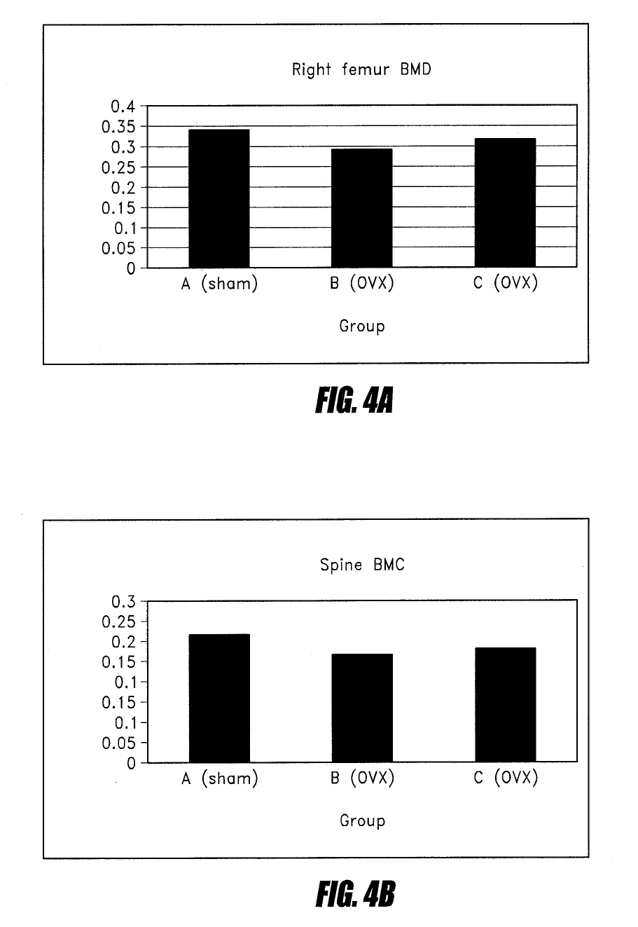

FIG. 4 shows the BMD of the right femur and spine after 16 weeks of feeding the fractions. In both cases the BMD of the control OVX group (Group B) is statistically significantly lower than that of the Sham Group A (statistically significant results are marked with an *), whereas the BMD of the group fed the acidic whey protein fraction (Group C) did not differ significantly from the Sham group, (p<0.05).

This experiment showed that OVX rats fed the control diet (Group B) lost significant amounts of bone in comparison to the Sham control rats (Group A), whereas surprisingly, the rats fed the acidic protein fraction (Group C) did not lose significant amounts of bone compared to the Sham rats. This showed that the acidic protein fraction of the invention can reduce or prevent the bone loss that occurs due to oestrogen deficiency.

The above describes some preferred embodiments of the present invention and indicates several possible modifications but it will be appreciated by those skilled in the art that other modifications can be made without departing from the scope of the invention.

We claim:

1. A method of maintaining or improving bone health comprising oral administration of an acidic protein fraction of whey, hydrolysates of an acidic protein fraction of whey or a combination thereof to a subject in need thereof, wherein the composition does not contain caseinoglycomacropeptide (CGMP).

2. A method of treating or preventing net bone loss comprising oral administration of an acidic protein fraction of whey, hydrolysates of an acidic protein fraction of whey or a combination thereof to a subject in need thereof, wherein the composition does not contain caseinoglycomacropeptide (CGMP).

3. A method of maintaining or improving bone health comprising oral administration of an isolated acidic protein fraction of whey, hydrolysates of the acidic protein fraction of acid whey or a combination thereof, wherein the fraction does not contain caseinoglycomacropeptide (CGMP), the fraction comprising proteins that have an isoelectric point of 4.9 or less and peptides generated from casein by the action of plasmin, the proteins comprising a protein with a molecular weight of approximately 65,000 Daltons.

4. A method of claim 1 wherein the fraction comprises proteins that have an isoelectric point of 4.9 or less; wherein the fraction comprises at least 70 % by weight of proteins of which at least 80 % by weight comprises osteopontin and proteose peptones.

5. A method of claim 1 wherein the acidic protein fraction comprises 70% by weight or more of proteins of which 80% by weight or more comprise osteopontin and proteose peptones.

6. A method of claim 1 wherein the proteins in the fraction have a molecular weight distribution of 3,000 to 65,000 as measured by SDS-PAGE.

7. A method of claim 5 wherein the proteose peptones comprise peptides generated from casein by the action of plasmin and include one or more of the proteins selected from the group comprising proteose peptone 5 (PP5), proteose peptone 8-slow (PP8-slow), proteose peptone 8-fast (PP8-fast), as well as the non-casein proteose peptone 3 (PP3).

8. A method of claim 1 wherein the sialic acid content of the fraction is in the range 0.8% to 6.5%.

9. A method of claim 1 wherein the phosphate content of the fraction is in the range 0.5% to 3%.

10. A method of claim 1 wherein the fraction is produced using anion exchange chromatography or strong anion exchange chromatography or a combination thereof.

11. A method of claim 1 wherein the fraction is derived from any one or more feedstocks selected from the group consisting of: recombined or fresh whole milk, recombined or fresh skim milk, reconstituted whole or skim milk powder, colostrum, milk protein concentrate (MPC), milk protein isolate (MPI), whey protein isolate (WPI), whey protein concentrate (WPC), whey, reconstituted whey powder, or derived from any milk processing stream, or derived from the permeates obtained by ultrafiltration and/or microfiltration of any one or more of these feedstocks.

12. A method of claim 1 wherein the fraction is derived from lactic acid whey or mineral acid whey.

13. A method of claim 1 wherein the fraction is produced by a method comprising the steps of:
(a) providing an aqueous solution that does not contain CGMP and that is derived from any one or more feedstocks selected from the group consisting of: recombined or fresh whole milk, recombined or fresh skim milk, reconstituted whole or skim milk powder, colostrum, milk protein concentrate (MPC), milk protein isolate (MPI), whey protein isolate (WPI), whey protein concentrate (WPC), whey, reconstituted whey powder, or derived from any milk processing stream, or derived from the permeates obtained by ultrafiltration and/or microfiltration of any one or more of these feedstocks;
(b) subjecting the aqueous solution to anion exchange chromatography at a pH of from about pH 3 to about pH 4.9;
(c) washing the anion exchange medium;
(d) eluting from the anion exchange medium an acidic protein fraction of whey.

14. A method of claim 13 wherein the aqueous solution provided in step (a) is or derived from lactic acid whey or mineral acid whey.

15. A method of claim 13 wherein the anion exchange chromatography is strong anion exchange chromatography.

16. A method of claim 13 wherein step (d) is carried out using NaCl or KCl a mixture thereof.

17. A method of claim 13 wherein step (d) is carried out using an acid or, two or more eluting solutions having different pHs.

18. A method of claim 13 wherein step (d) is carried out using an eluting solution having a pH between 5.0 and 9.0 and a salt concentration up to 1.0 M.

19. A method of claim 1 wherein the fraction is administered with a physiologically acceptable amount of calcium, magnesium, vitamin C, vitamin D, vitamin E, vitamin K₂ or zinc.

20. A method of claim 1 wherein the fraction comprises one or more of osteopontin, bone sialoprotein, proteose peptone 3, proteose peptone 5, proteose peptone 8, sialyated and phosphorylated proteins and peptides obtained therefrom, and alpha-s1-casein phosphopeptides.
